(19) United States
(12) Patent Application Publication
(10) Pub. No.: US 2004/0162540 A1
(43) Pub. Date: Aug. 19, 2004

(54) Transfer device

(75) Inventors: Matthew Walenciak, Westfield, NJ (US); Frank Augello, Cedar Knolls, NJ (US); Kirk Swenson, North Caldwell, NJ (US)

Correspondence Address: Kirk M. Miles, Esq., WEBB ZIESENHEIM LOGSDON ORKIN & HANSON, P.C., 700 Koppers Building, 436 Seventh Avenue, Pittsburgh, PA, 15219-1818, US

(73) Assignee: Becton, Dickinson and Company

(21) Appl. No.: 10/365,939

(22) Filed: Feb. 13, 2003

(51) Int. Cl. A61M 5/32; A61B 19/00

(52) U.S. Cl. 604/411

(57) ABSTRACT

The present invention is directed to a fluid transfer device including first and second sampling tube holders for accommodating sampling tubes therein, with the holders joined together through a coupler. The coupler includes a cannula having a first end extending into the first sampling tube holder and a second end extending into the second sampling tube holder, thereby establishing a path for fluid flow between a first sampling tube accommodated within the first holder and a second sampling tube accommodated within the second holder through the cannula. The coupler may be a two piece system including two hubs with separate cannulas extending from each hub, and each hub may be separately joined to the holders prior to joining the hubs to form the coupler. The coupler may further include a connector extending between the two hubs.

### BACKGROUND OF THE INVENTION

1. Field of the Invention

The present invention relates to a fluid transfer assembly. More particularly, the present invention is directed to a medical fluid transfer device that links two blood collection tube holders.

2. Description of Related Art

Disposable medical devices having medical needles are used for withdrawing fluid from the body of a patient. When necessary, additional medical devices having medical needles are used to transfer all or part of the withdrawn fluid from one collection container to another container. Such disposable medical devices typically include blood-collecting needles, fluid handling needles and assemblies thereof. Current medical practice requires that fluid containers and needle assemblies used in such devices be inexpensive and readily disposable. Consequently, existing blood collection and transfer devices typically employ some form of durable, reusable holder on which detachable and disposable medical needles and fluid collection tubes may be mounted. A blood collection or transfer device of this nature may be assembled prior to use and then disassembled after use. Thus, these blood collection and transfer devices allow repeated use of a relatively expensive holder upon replacement of relatively inexpensive medical needles and/or fluid collection tubes. In addition to reducing the cost of collecting and transferring blood specimens, these blood collection and transfer devices help minimize the production of hazardous waste material.

One particular example of a system for the transfer and collection of body fluids, i.e., blood, is described in U.S. Pat. No. 5,360,423. This patent describes a needle assembly attached to a port guide. The needle assembly includes a needle surrounded by a piercable sheath. The needle assembly may include a female or a male connector for interconnection with a separate member, for example, a conventional syringe male fitting.

The current art does not provide for a convenient device that allows for the direct transfer of a fluid, i.e., blood, from one sampling container to another. Current devices and assemblies require intermediate steps to move the fluid from the first container to the second.

Accordingly, a need exists for a fluid transfer device in which fluid from one container, for example, a blood collection tube, is directly transferred to a second container, for example, a second blood collection tube.

### SUMMARY OF THE INVENTION

The present invention is directed to a medical fluid transfer device which involves the linking of two conventional blood collection sampling tube holders, or needle holders, which are typically used for blood collection and sampling procedures. The holders are arranged in opposing relation, such as with the threaded ends opposing each other, and are coupled together through a coupling unit or coupler. The coupler includes pointed needle cannulae extending from both ends of the coupler and desirably includes a piercable sleeve encompassing each of the needle cannulae. The coupler also includes structure for mating with both holders, such as external threads on both ends for threaded engagement with corresponding threads on the two holders. The coupler thereby links two holders together, with one needle cannula extending within each of the holders. As such, fluids can be transferred from one container to a second container, both of which include piercable membranes, such as evacuated blood collection tubes, by inserting each of the containers into one of the holders and piercing the piercable membranes with the respective needle cannula. Moreover, the transfer system can be set up to transfer an exact amount of fluid based on an amount of vacuum within the container receiving the transferred sample.

The coupler has a variety of assembly arrangements. For example, the coupler may be constructed of a one-piece hub, with a double-ended needle cannula mounted therethrough, and desirably with structure on both ends thereof for mating with each of the holders, such as threads. Alternatively, the coupler may be a two-piece hub, with mating structure such as threads on each hub and with a separate needle cannula extending from each hub, with the two-piece hub mated together through a male/female luer-type taper engagement. As such, the sampling tube holders can be disengaged through the two-piece coupler. Further, the coupler may be a three-piece hub, with two separate hubs including respective needle cannulae extending therefrom and a connector extending between the two hubs. The two hubs may each include a male luer taper with the connector having opposing female tapering surfaces to provide a male-to-male luer connector, or the two hubs may each include a female luer taper, with the connector having opposing male tapering surfaces to provide a female-to-female luer connector. The coupler may also be a three-piece hub in which two hubs including separate needle cannulae are interconnected through a flexible tube-like structure.

The present invention is also directed to a method of transferring body fluids between a first sampling tube and a second sampling tube. The method involves providing a fluid transfer device including first and second sampling tube holders for accommodating first and second sampling tubes therein. The first and second sampling tube holders are joined together through a coupler which includes a cannula having a first puncture tip and a second puncture tip at opposing ends such that the first puncture tip extends into the first sampling tube holder and the second puncture tip extends into the second sampling tube holder. The second puncture tip further includes a piercable elastomeric sleeve extending thereabout. A first sampling tube having a puncturable closure is inserted within the first sampling tube holder, and the first puncture tip punctures or pierces the closure of the first sampling tube within the first holder. The piercable sleeve extending about the second puncture tip acts as a valve to maintain the contents of the first sampling tube therein. A second sampling tube having a puncturable closure is inserted within the second sampling tube holder and the second puncture tip punctures or pierces the closure of the second sampling tube within the second holder. As such, fluid flow is established between the first sampling tube and the second sampling tube through the cannula, thereby transferring fluid between the first sampling tube and the second sampling tube.

The method may further include providing and joining the first and second sampling tube holders through the coupler. The coupler may be any of the embodiments noted. Desirably, the coupler includes a first hub and a second hub and the cannula includes a first cannula and a second cannula, with the first cannula extending from the first hub and the second cannula extending from the second hub. The first sampling tube holder may be joined with the first hub with the first puncture tip extending within the first sampling tube holder, and the second sampling tube holder may be joined with the second hub with the second puncture tip extending within the second sampling tube holder. The two holders as such may be mated through the first and second hubs.

In a further embodiment, the present invention is directed to a method of assembling a fluid transfer device. The method includes providing first and second sampling tube holders for accommodating first and second sampling tubes therein, and joining the holders through a coupler including a cannula having a first puncture tip and a second puncture tip at opposing ends, such that the first puncture tip extends into the first sampling tube holder and the second puncture tip extends into the second sampling tube holder. Desirably, the coupler includes a first hub and a second hub and the cannula includes a first cannula and a second cannula, with the first cannula extending from the first hub and the second cannula extending from the second hub. The first sampling tube holder may be joined with the first hub with the first puncture tip extending within the first holder and the second sampling tube holder may be joined with the second hub with the second puncture tip extending within the second holder. The first and second hubs can then be mated to join the first and second holders. The method may further include chemically bonding the first hub with the second hub. Further, the method may include packaging and/or sterilizing the fluid transfer device as an assembly with first and second sampling tube holders joined through the coupler.

In yet a further embodiment, the present invention relates to a kit of parts, including first and second sampling tube holders. The first and second sampling tube holders each include a first open end for accommodating a sampling tube therethrough and a second end having an opening therethrough. Also included is a coupler for joining the first sampling tube holder and the second sampling tube holder. The coupler includes a hub having an internal passageway extending between a first end and a second end. A cannula extends from the internal passageway at the first end of the hub to form a first puncture tip and at the second end to form a second puncture tip. Each puncture tip further includes a piercable sleeve extending thereabout. Each end of the coupler includes structure for engagement with the second ends of the first and second sampling tube holders, to provide the first and second puncture tips extending through the openings at the second ends of the respective sampling tube holders.

The present invention also provides a kit of parts which includes separate first and second sampling tube holders, each having a hollow interior, a first open end for accommodating a sampling tube therein, and a second end supporting a cannula extending therethrough with a puncture tip extending into the hollow interior of the respective holder. Each of the sampling tube holders includes structure for engagement with the other sampling tube holder at the second end thereof, for providing a path for fluid flow between the cannula of the first sampling tube holder and the cannula of the second sampling tube holder.

For example, the second end of the first sampling tube holder may include a hollow male tapering surface and the second end of the second sampling tube holder may include a hollow female tapering surface for frictional mating therebetween. Alternatively, the second end of both of the sampling tube holders includes hollow female tapering surfaces, and the kit of parts further includes a hollow connector having opposing first and second ends, each with a male tapering surface. The male tapering surface of the first end of the hollow connector is adapted for frictional mating with the female tapering surface of the first sampling tube holder and the male tapering surface of the second end of the hollow connector is adapted for frictional mating with the female tapering surface of the second sampling tube holder. As such, a path for fluid flow can be established between the first cannula and the second cannula through the hollow connector when the parts are assembled.

### BRIEF DESCRIPTION OF THE DRAWINGS

FIG. 1 is perspective view of a fluid transfer device in accordance with a first embodiment of the present invention;

FIG. 2 is an exploded perspective view of the fluid transfer device of FIG. 1;

FIG. 3 is a side cross-sectional view of the fluid transfer device of FIG. 1;

FIG. 4 is perspective view of a fluid transfer device in accordance with an alternate embodiment of the present invention;

FIG. 5 is an exploded perspective view of the fluid transfer device of FIG. 4;

FIG. 6 is a side cross-sectional view of the fluid transfer device of FIG. 4;

FIG. 7 is perspective view of a fluid transfer device in accordance with a further embodiment of the present invention;

FIG. 8 is an exploded perspective view of the fluid transfer device of FIG. 7;

FIG. 9 is a side cross-sectional view of the fluid transfer device of FIG. 7;

FIG. 10 is perspective view of a fluid transfer device in accordance with a further embodiment of the present invention;

FIG. 11 is an exploded perspective view of the fluid transfer device of FIG. 10;

FIG. 12 is a side cross-sectional view of a fluid transfer device of FIG. 10;

FIG. 13 is perspective view of a fluid transfer device in accordance with yet a further embodiment of the present invention;

FIG. 14 is a side cross-sectional view of the fluid transfer device of FIG. 13; and

FIG. 15 is a side cross-sectional view of a fluid transfer device of FIG. 1 shown in use during a transfer procedure.

### DESCRIPTION OF THE PREFERRED EMBODIMENTS

While this invention is satisfied by embodiments in many different forms, there is shown in the drawings and will herein be described in detail, the preferred embodiments of the invention, with the understanding that the present disclosure is to be considered as exemplary of the principles of the invention and is not intended to limit the invention to the embodiments illustrated. Various other modifications will be apparent to and readily made by those skilled in the art without departing from the scope and spirit of the invention. The scope of the invention will be measured by the appended claims and their equivalents.

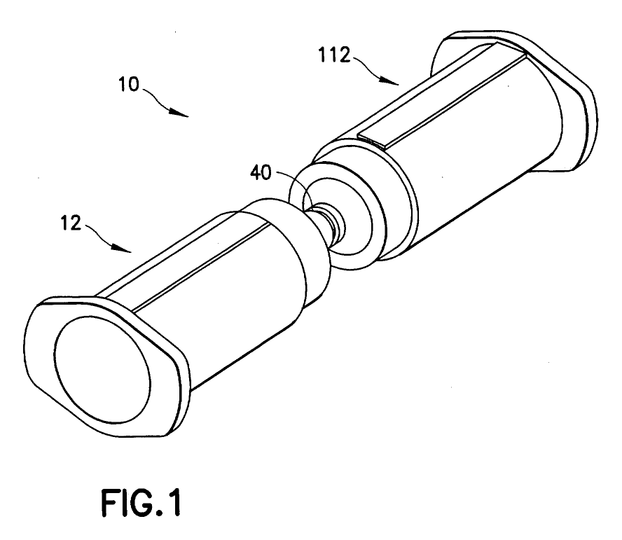

Referring to the drawings in which like reference characters refer to like parts throughout the several views thereof, FIG. 1 illustrates a fluid transfer device including a first and second sampling tube holder and a coupler, in accordance with the present invention and the related features. The present invention is generally described in terms of a blood transfer device including a coupler for linking a first sampling tube holder with a second sampling tube holder and is directed to such an assembly, as well as to the coupler itself and subassemblies incorporating the coupler.

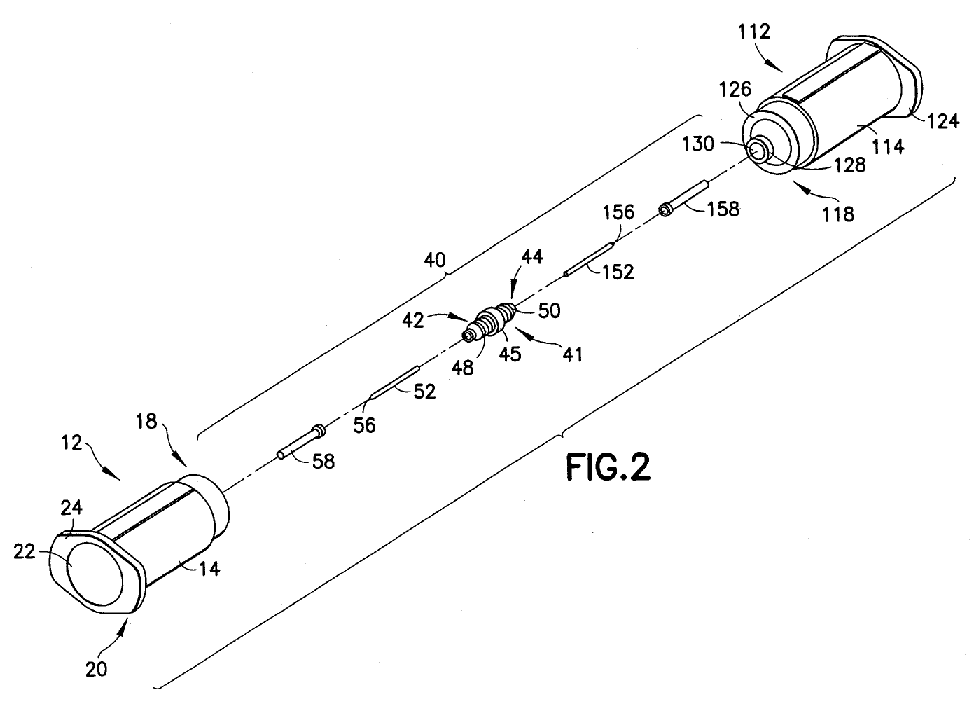

FIGS. 1-3 illustrate a fluid transfer device 10 having a first sampling tube holding region such as a first sampling tube holder 12, and a second sampling tube holder region such as a second sampling tube holder 112, which are linked through a coupler 40. Desirably, the first sampling tube holder region and the second sampling tube holder region are provided through a pair of connected conventional holders which are known for holding a sampling needle and a sampling tube during a phlebotomy procedure.

Each of first sampling tube holder 12 and second sampling tube holder 112 includes a generally tubular or cylindrical housing 14, 114 defined by a tubular wall 16, 116 extending between a forward end 18, 118 and a rearward end 20, 120 with an internal opening 22, 122 extending therethrough. Rearward end 20, 120 of housing 14, 114 is open-ended into internal opening 22, 122 and may include a flange 24, 124 extending perimetrically outward around the open end at rearward end 20, 120. Forward end 18, 118 of housing 14, 114 includes a forward wall 26, 126 which extends to a shoulder to form a cylindrical neck 28, 128. Neck 28, 128 includes an aperture, such as an opening 30, 130, therethrough which extends into internal opening 22, 122 of housing 14, 114. Structure or means for engagement with coupler 40, 140 to maintain such a fluid transfer device during a blood transfer procedure is further provided at forward end 18, 118. For example, internal threads 32, 132 may be provided within the opening 30, 130 of neck 28, 128, which are provided for threaded engagement with corresponding threads on coupler 40, 140. Alternatively, any structure or means for engagement which is capable of connecting coupler 40, 140 to first sampling tube holder 12 and second sampling tube holder 112 may be provided, such as a snap-fit engagement, releasable engagement, and other connections.

First sampling tube holder 12 and second sampling tube holder 112 are particularly sized so as to accommodate respective first and second sampling tubes having a maximum diameter of about 16 millimeters, desirably between about 13 millimeters and 16 millimeters. First sampling tube holder 12 and second sampling tube holder 112 may be constructed of any material known in the art, desirably a polymeric material. Desirably, first sampling tube holder 12 and second sampling tube holder 112 are constructed of polypropylene. One particular example of a sampling tube holder which is particularly useful is the VACUTAINER® brand holder available from Becton, Dickinson and Company of Franklin Lakes, N.J.

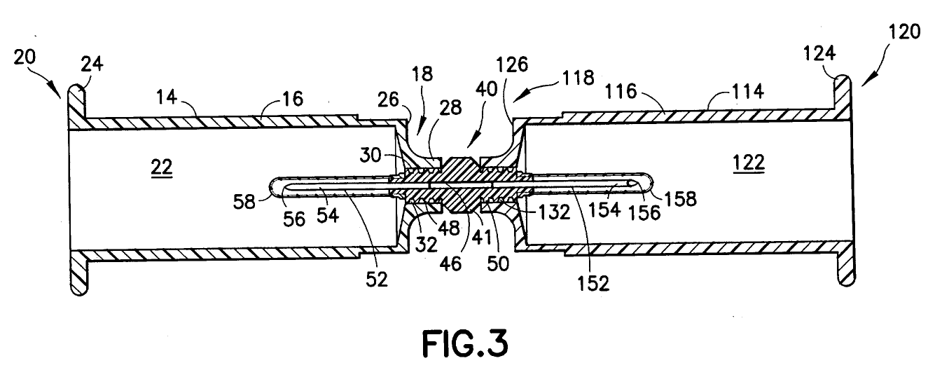

First and second sampling tube holders 12 and 112 are joined together in an end-to-end fashion through coupler 40, as shown in FIGS. 1 and 3. Coupler 40 is defined by housing 41, and includes a first end 42 and a second end 44 which are separated by a flange 45 therebetween, and with a passageway 46 extending therethrough establishing a path for fluid flow. Each of first end 42 and second end 44 include structure or means for engagement with first sampling tube holder 12 and second sampling tube holder 112, respectively. For example, external threads 48, 50 may be provided at first end 42 and second end 44, respectively, for threaded engagement with corresponding internal threads 32 of first sampling tube holder 12 and internal threads 132 of second sampling tube holder 112. Alternatively, any structure or means for engagement which is capable of connecting coupler 40 to first sampling tube holder 12 and second sampling tube holder 112 may be provided, such as a corresponding surface for snap-fit engagement, releasable engagement, and other connections.

First end 42 also includes a first hollow needle cannula 52 having an internal lumen 54 extending therethrough. First needle cannula 52 extends away from coupler 40 at first end 42 along a common axis with passageway 46. When first end 42 engages first sampling tube holder 12, first needle cannula 52 extends into first sampling tube holder 12 through opening 30. First needle cannula 52 further includes a sharp puncture tip 56 typically provided for puncturing a stopper or septum of a collection tube when first end 42 of coupler 40 engages first sampling tube holder 12. Desirably, a piercable elastomeric sleeve or sheath 58 extends from first end 42 and about the end of first needle cannula 52 thereby encompassing the puncture tip 56. As will be discussed in more detail herein in terms of use of the fluid transfer device, this sleeve or sheath 58 acts as a valve for preventing the contents of the sampling tubes from being expelled. In addition, sleeve or sheath 58 functions as a safety mechanism for protection from puncture tip 56.

Likewise, second end 44 includes a second hollow needle cannula 152 having an internal lumen 154 extending therethrough. Second needle cannula 152 extends away from coupler 40 at second end 44 along a common axis with passageway 46. When second end 44 engages second sampling tube holder 112, second needle cannula 152 extends into second sampling tube holder 112 through opening 130. Second needle cannula 152 further includes a sharp puncture tip 156 in a similar manner as first needle cannula 52, which is provided for puncturing a stopper or septum of a collection tube when second end 42 of coupler 40 engages second sampling tube holder 112. As with first needle cannula 52, a piercable elastomeric sleeve or sheath 158 may extend from second end 42 of coupler 40 and about the end of second needle cannula 152 thereby encompassing the puncture tip 156. Sleeve or sheath 158 acts as a valve as well as a safety mechanism for protection from puncture tip 156 in a similar manner as discussed above.

First needle cannula 52 and second needle cannula 152 may be provided as distinct and separate members, so long as fluid flow is established between the respective lumen, for example, via passageway 46 of coupler 40. Alternatively, first needle cannula 52 and second needle cannula 152 may be provided as a single needle cannula extending through passageway 46 of coupler 40, with first puncture tip 56 and second puncture tip 156 extending respectively from first end 42 and second end 44 of coupler 40.

First needle cannula 52 and second needle cannula 152 may be attached to coupler 40 by mechanical engagement or through adhesive means. Regardless of the attachment mechanism, the force required for first needle cannula 52 and/or second needle cannula 152 to pierce a stopper of a blood container or collection tube is less than the force required to unattach first needle cannula 52 and/or second needle cannula 152 from coupler 40.

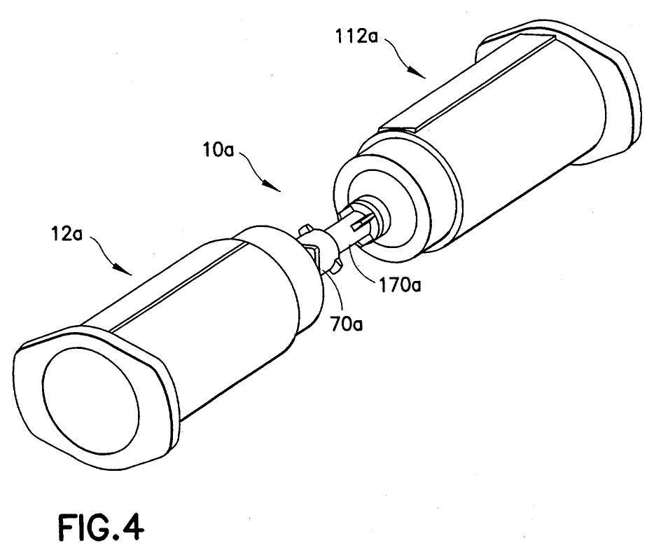

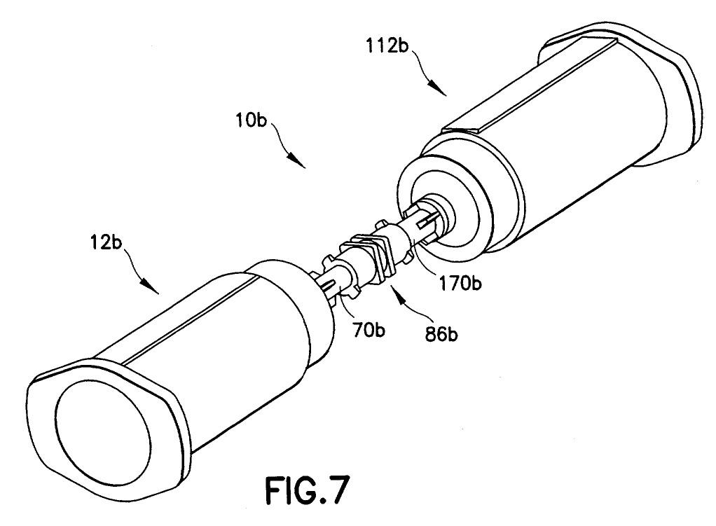

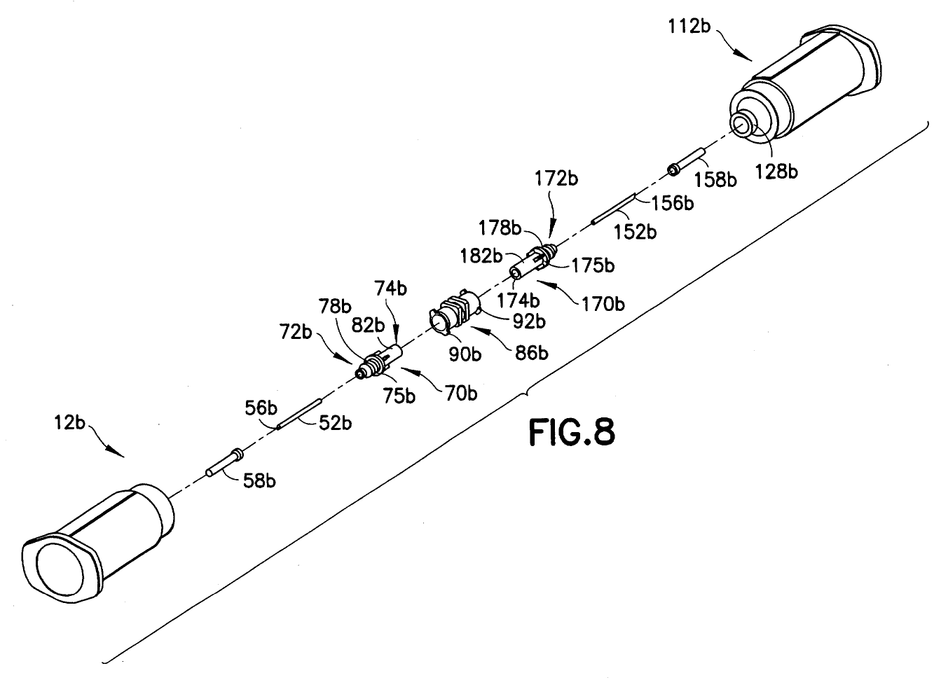

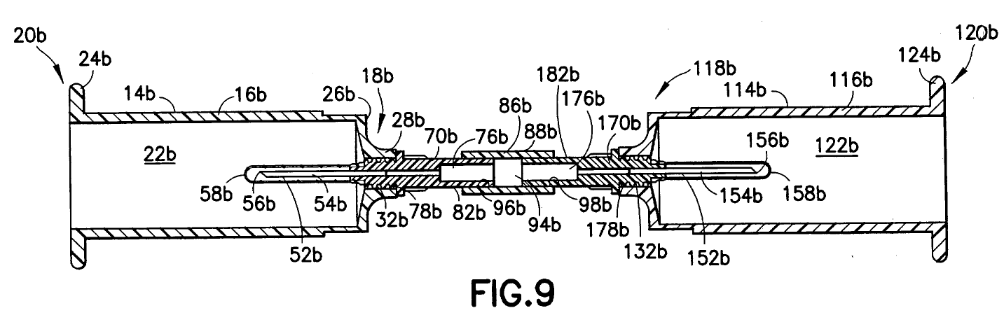

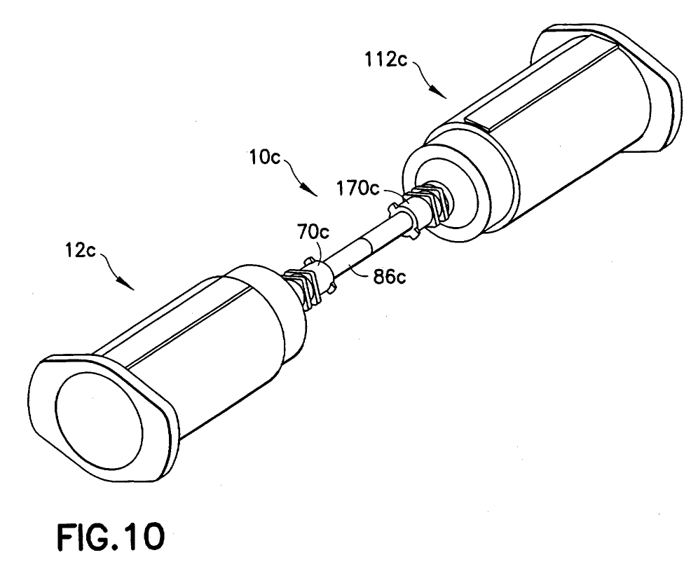

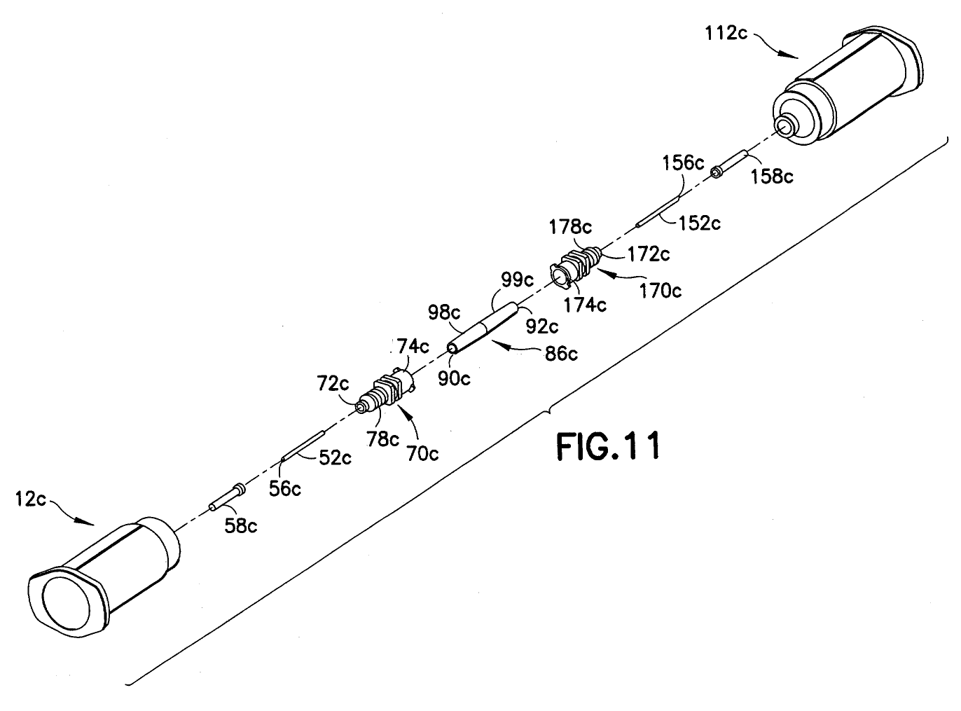

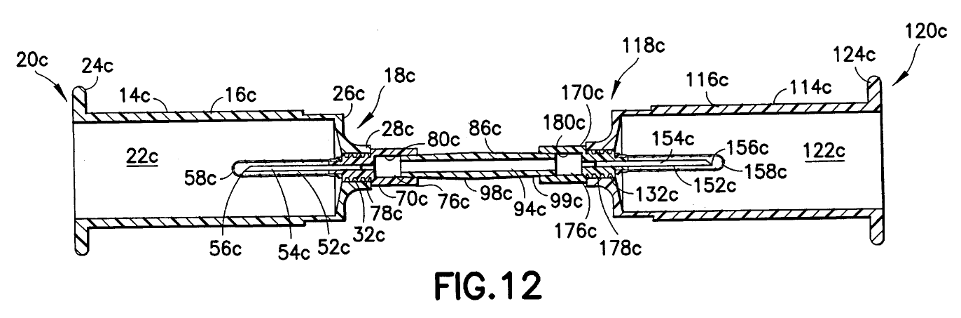

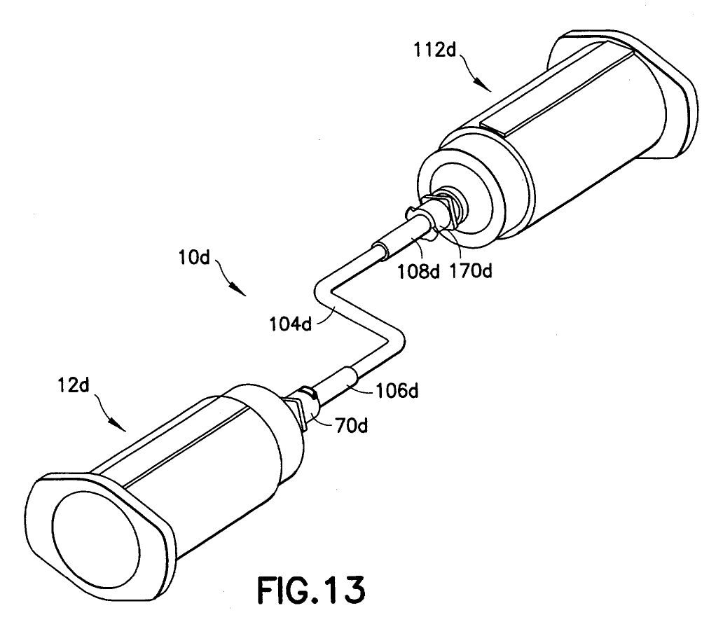

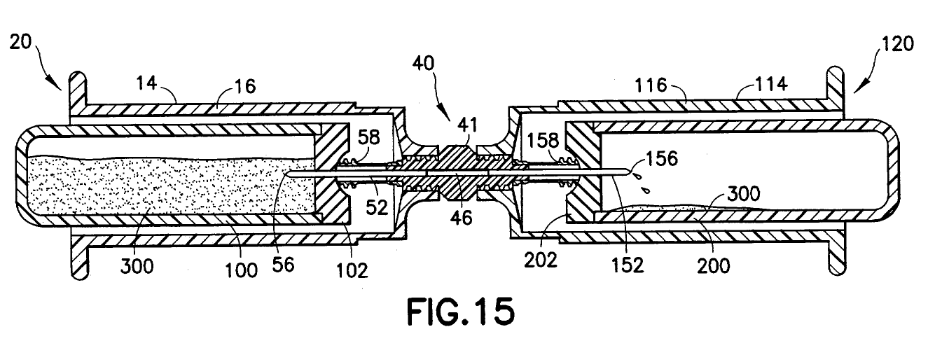

FIGS. 4-15 depict further embodiments of the present invention which include many components that are substantially identical to the components of FIGS. 1-3. Accordingly, similar components performing similar functions will be numbered identically to those components of FIGS. 1-3, except that a suffix “a” will be used to identify those similar components in FIGS. 4-6, a suffix “b” will be used to identify those similar components in FIGS. 7-9, a suffix “c” will be used to identify those similar components in FIGS. 10-12, and a suffix “d” will be used to identify those similar components in FIGS. 13-15.

FIGS. 4-6 illustrate a fluid transfer device 10a where the coupler 40a includes a first hub 70a and a second hub 170a. First hub 70a includes holder end 72a for engagement with first sampling tube holder 12a, and a connector end 74a for mating with second hub 170a. A flange 75a may be provided between holder end 72a and connector end 74a. An internal passageway 76a extends through first hub 70a between holder end 72a and connector end 74a. Holder end 72a further includes structure for mating with first sampling tube holder 12a, such as external threads 78a.

Second hub 170a includes holder end 172a for engagement with second sampling tube holder 112a, and connector end 174a for mating with first hub 70a, with flange 175a provided between holder end 172a and connector end 174a. An internal passageway 176a extends through second hub 170a between holder end 172a and connector end 174a in a similar manner as with first hub 70a. Holder end 172a further includes structure for mating with second sampling tube holder 112a, such as external threads 178a.

In such an embodiment in which the coupler is provided as two separate components in the form of first hub 70a and second hub 170a, first needle cannula 52a is associated with first hub 70a and second needle cannula 152a is associated with second hub 170a. As such, first needle cannula 52a extends from holder end 72a of first hub 70a, for extension through opening 30a and within internal opening 22a of first holder 12a when first hub 70a is mated with first holder 12a. Likewise, second needle cannula 152a extends from holder end 172a of second hub 170a, for extension through opening 130a and within internal opening 122a of second holder 112a when second hub 170a is mated with second holder 112a. Elastomeric sleeves or sheaths 58a and 158a extend from respective ends of first and second hubs 70a, 170a to encompass the respective first and second puncture tips 56a, 156a, in a similar manner as discussed above.

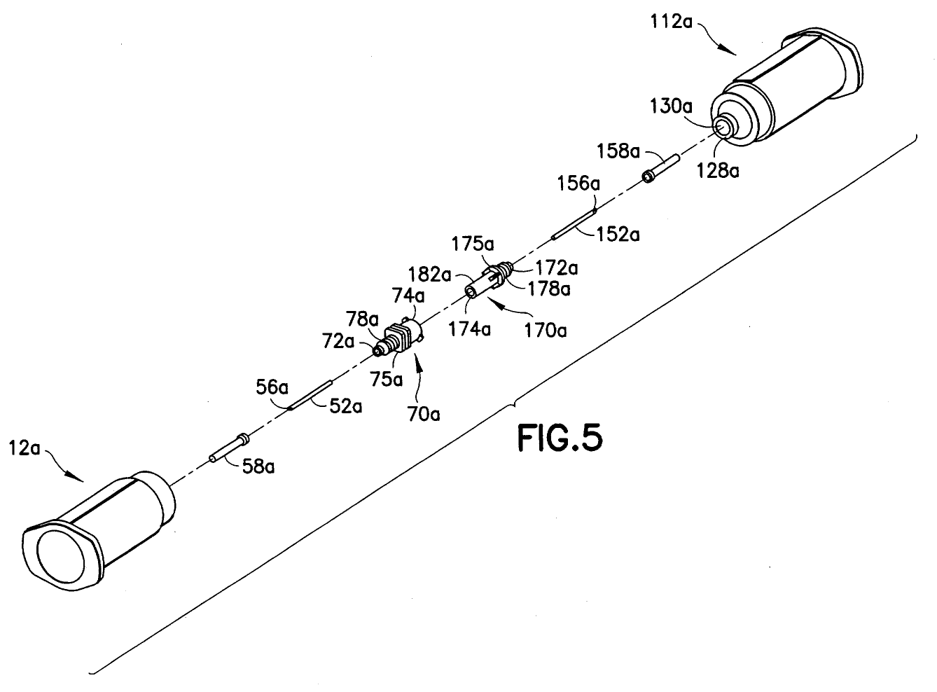

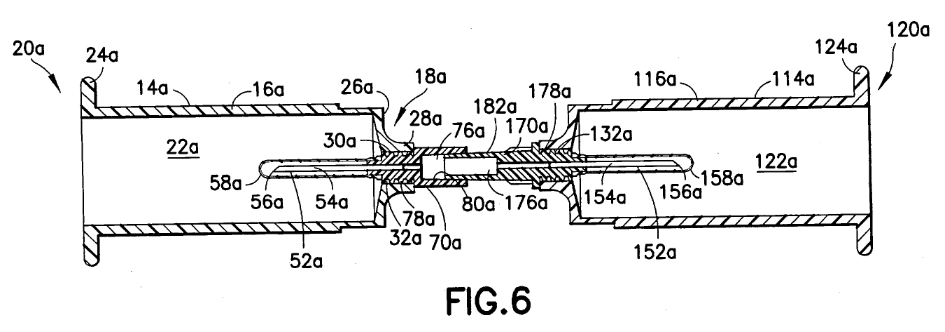

Desirably, connector end 74a of first hub 70a and connector end 174a of second hub 170a include structure for mating engagement with each other. For example, one of first hub 70a or second hub 170a may include a male taper surface while the other may include a female taper surface, for establishing a luer-type engagement therebetween. In particular, as shown in FIGS. 5 and 6, first hub 70a may be provided with a female taper surface 80a, while second hub 170a is provided with a male taper surface 182a. Female taper surface 80a is capable of accommodating male taper surface 182a therein, thereby providing structure for mating engagement between first hub 70a and second hub 170a. Female taper surface 80a and male taper surface 182a are engageable, for example, in a friction fit to attach first hub 70a and second hub 170a in a frictional engagement therebetween. Alternatively or in addition to the frictional engagement, an adhesive may be provided between female taper surface 80a and male taper surface 182a.

FIGS. 7-9 illustrate a fluid transfer device 10b in a further embodiment, in which coupler 40b includes a first hub 70b and a second hub 170b in a similar manner as in the embodiment of FIGS. 4-6, but with the respective connector ends 74b, 174b both including a male taper surface 82b, 182b, respectively. As such, coupler 40b in FIGS. 7-9 further includes a connector 86b for establishing connection between first hub 70b and second hub 170b. In particular, connector 86b is defined by a tubular wall 88b extending between a first end 90b and a second end 92b, with a passageway 94b extending therethrough. First end 90b of connector 86b includes a female taper surface 96b, and second end 92b of connector 86b also includes a female taper surface 97b. As such, connector 86b as shown in FIGS. 7-9 provides for a male-to-male connection, such as for connecting male taper surface 82b of first hub 70b and male taper surface 182b of second hub 170b, thereby establishing fluid flow therebetween through passageway 94b.

FIGS. 10-12 illustrate a further embodiment of a fluid transfer device 10c which is similar to the embodiment depicted in FIGS. 7-9, but with the coupler including a female-to-female type connector instead of the male-to-male type connector of FIGS. 7-9. In particular, coupler 40c includes a first hub 70c and a second hub 170c in a similar manner as described above, but with the respective connector ends 74c, 174c both including a female taper surface 82c, 182c, respectively. Coupler 40c in FIGS. 10-12 therefore further includes a connector 86c for establishing connection between first hub 70c and second hub 170c. Connector 86c is defined by a tubular wall 88c extending between a first end 90c and a second end 92c, with a passageway 94c extending therethrough. First end 90c of connector 86c includes a male taper surface 98c, and second end 92c of connector 86c also includes a male taper surface 99c. As such, connector 86c as shown in FIGS. 10-12 provides for a female-to-female connection, such as for connecting female taper surface 80c of first hub 70c and female taper surface 180c of second hub 170c, thereby establishing fluid flow therebetween through passageway 94c.

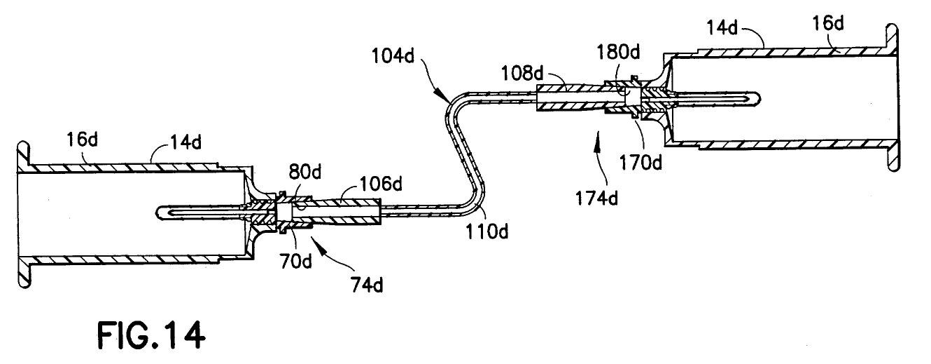

FIGS. 13-14 depict yet a further embodiment of a fluid transfer device 10d which is similar to the previous embodiments, but with the coupler including a flexible tubing extending between first hub 70d and second hub 170d, instead of a connector. Flexible tubing 104d extends between a first end 106d and a second end 108d, with an internal passageway 110d extending therethrough. First end 106d is attached to connector end 74d of first hub 70d, while second end 108d is attached to connector end 174d of second′hub 170d. Desirably, connector ends 74d, 174d, of first and second hubs 70d, 170d, are female taper surfaces 80d and 180d, respectively, thereby providing a tapering surface for engagement with the respective ends of flexible tubing 104d. Flexible tubing 104d provides for fluid passage between first hub 70d and second hub 170d.

The present invention further encompasses a method of assembling a fluid transfer device as well as a method of transferring fluids using a fluid transfer device. Accordingly, a fluid transfer device according to any of the above-described embodiments may be provided for use in a pre-packaged and pre-sterilized form. Alternatively, a fluid transfer device according to any of the above-described embodiments may be assembled, such as by joining the first sampling tube holder and the second sampling tube holder through a coupler in any of the forms described above. Desirably, the first sampling tube holder and the second sampling tube holder are mechanically or chemically bonded to the coupler, for example by sonically welding or adhesively attaching the first sampling tube holder to the holder end of a first hub and the second sampling tube holder to the holder end of a second hub. As such, the first sampling tube holder can be provided with the first hub attached thereto, and the second sampling tube holder can be provided with the second hub attached thereto. The first and second sampling tube holders can thereafter be mated and joined through the first and second hubs, such as through frictional engagement between the respective connector surfaces, or by mating and sonically welding or adhesively attaching the connector ends of the first and second hubs. Moreover, a connector may be provided between the connector ends of the first and second hubs, as discussed with respect to the alternate embodiments described above.

Desirably, the assembly is assembled, packaged and sterilized as a single unit, to provide an assembly for use as a fluid transfer device. It is further contemplated that the components can be assembled in sub-assemblies, such as with the first hub attached to the first sampling tube holder and the second hub attached to the second sampling holder, with the sub-assemblies being joined together just prior to use, such as by frictional engagement therebetween or through a connector.

In use, the fluid transfer assembly is provided as described with respect to any of the above-referenced embodiments, although use of the invention will be described generally in terms of the embodiment of FIGS. 1-3 and 15, with first sampling tube holder 12 attached to second sampling tube holder 112 through coupler 40. With fluid transfer device 10 provided as such, two separate sampling tubes 100, 200, each having a puncturable closure or stopper 102, 202, are provided for transfer of a fluid therebetween. One of the sampling tubes contains a fluid 300 such as blood therein, for transfer to the other sampling tube. The first sampling tube 100 containing the sample fluid 300 therein is inserted into internal opening 22 of first sampling tube holder 12 through rearward end 20 thereof, while the second sampling tube 200 is inserted into internal opening 122 of second sampling tube holder 112 through rearward end 120 thereof.

With fluid transfer device 10 assembled, first puncture tip 56 of first needle cannula 52 extends within internal opening 22 of first sampling tube holder 12, and second puncture tip 156 of second needle cannula 152 extends within internal opening 122 of second sampling tube holder 112. The first sampling tube 100 is inserted into first sampling tube holder 12 to cause the stopper 102 closing the end of the first sampling tube 100 to contact and displace elastomeric sleeve or sheath 58. Further insertion of the first sampling tube 100 within first sampling tube holder 12 causes first puncture tip 56 to puncture the stopper 102 and causes first needle cannula 52 to be inserted into the interior of the first sampling tube 100 in fluid engagement with the sample fluid 300 present therein. At this point, the elastomeric sleeve or sheath 158 which extends about second needle cannula 152 acts as a valve, preventing the convents of first sampling tube 100 from being expelled through passageway 46 and into the ambient environment. The stopper 202 of second sampling tube 200 is then punctured by second puncture tip 156 in a similar manner through displacement of sheath 158 and insertion of second puncture tip 156 through stopper 202, such that second needle cannula 152 is in fluid engagement with the interior of the second sampling tube 200. Since first needle cannula 52 and second needle cannula 152 are in fluid communication therebetween, such as through passageway 46 of coupler 40, and since first needle cannula 52 now extends within the first sampling tube 100 and since second needle cannula 152 now extends within the second sampling tube 200, a path for fluid flow is established between the first and second sampling tubes 100, 200 through fluid transfer device 10.

Desirably, the second sampling tube 200 into which the fluid sample is to be transferred is evacuated at a pressure below that of the first sampling tube 100 containing the sample fluid 300 to be transferred. As such, the negative pressure within the second sampling tube 200 causes fluid flow from the first sampling tube 100, through first and second cannulas 52 and 152, and into the second sampling tube 200 automatically upon insertion of second needle cannula 152 into the second sampling tube 200.

The amount of fluid transferred between the tubes can be controlled by the length of time of transfer or by the amount of vacuum in the second sampling tube. It is contemplated that the pressure within the second sampling tube can be fixed at a specific value in order to transfer a predetermined quantity of fluid from the first sampling tube into the second sampling tube. As such, the exact pressure value will be dependent upon the amount and type of fluid being transferred, and, thus, can be controlled during preparation of the empty sampling tubes, i.e., during manufacturing.

Upon transfer of the desired amount of fluid, the first and second sampling tubes can be removed from the respective first and second sampling tube holders 12, 112 and stored for appropriate analytical procedures. Fluid transfer device 10 can then be appropriately discarded.

By providing the fluid transfer device as described herein, fluids can be safely and conveniently transferred between sample containers, and in particular between evacuated sample tubes. Moreover, in many instances it is desirable to draw a partial sample from a sample after it has been collected. For example, in certain instances it is desirable to collect a blood sample and to allow the sample to begin to coagulate, and to then sample a portion of that blood sample for further analysis. The present invention provides an effective mechanism and method for this particular procedure, without requiring transfer between a secondary container or syringe, and without risk of contamination through a pouring off procedure.

It will be understood by those skilled in the art that while the foregoing description sets forth in detail preferred embodiments of the present invention, modifications, additions, and changes might be made thereto without departing from the spirit and scope of the invention.

The invention claimed is:

1. A fluid transfer device comprising:
a first sampling tube holder region having an internal opening capable of accommodating a first sampling tube therein;
a second sampling tube holder region having an internal opening capable of accommodating a second sampling tube therein; and
a coupler joining said first sampling tube holder region and said second sampling tube holder region, said coupler including an internal fluid path extending between a first end and a second end, said first end including a first puncture tip extending into said internal opening of said first sampling tube holder region and said second end including a second puncture tip extending into said internal opening of said second sampling tube holder region,
wherein said fluid transfer device is capable of establishing a path for fluid flow between a first sampling tube accommodated within said internal opening of said first sampling tube holder region and a second sampling tube accommodated within said internal opening of said second sampling tube holder region through said coupler.

2. The fluid transfer device according to claim 1, wherein:
said coupler comprises a hub having an internal passageway extending between a first end and a second end with a cannula extending from said internal passageway at said first end of said hub to form said first puncture tip and at said second end of said hub to form said second puncture tip.

3. The fluid transfer device according to claim 2, wherein said cannula is attached within said internal passageway of said hub by mechanical or adhesive means.

4. The fluid transfer device according to claim 2, wherein said first sampling tube holder region and said second sampling tube holder region are distinct first and second sampling tube holders, and wherein said first end of said hub includes structure for engagement with said first sampling tube holder and said second end of said hub includes structure for engagement with said second sampling tube holder.

5. The fluid transfer device according to claim 4, wherein:
said first sampling tube holder includes internal threads and said first end of said hub includes external threads for engagement with said internal threads of said first sampling tube holder, and
said second sampling tube holder includes internal threads and said second end of said hub includes external threads for engagement with said internal threads of said second sampling tube holder.

6. The fluid transfer device according to claim 4, wherein said first end of said hub is attached to said first sampling tube holder such that the force required for said first end of said cannula to pierce a stopper of a first sampling tube accommodated within said internal opening of said first sampling tube holder is less than the force required to unattach said first end of said hub from said first sampling tube holder, and said second end of said hub is attached to said second sampling tube holder such that the force required for said second end of said cannula to pierce a stopper of a second sampling tube accommodated within said internal opening of said second sampling tube holder is less than the force required to unattach said second end of said hub from said second sampling tube holder.

7. The fluid transfer device according to claim 4, wherein said first end of said hub is adhesively attached to said first sampling tube holder and said second end of said hub is adhesively attached to said second sampling tube holder.

8. The fluid transfer device according to claim 2, further comprising:
a first elastomeric sleeve extending about a first end of said cannula and within said internal opening of said first sampling tube holder; and
a second elastomeric sleeve extending about a second end of said cannula and within said internal opening of said second sampling tube holder.

9. The fluid transfer device according to claim 2, wherein said cannula comprises a single member.

10. The fluid transfer device according to claim 2 wherein a first end of said cannula and a second end of said cannula comprise separate first and second cannulas in fluid communication therebetween.

11. The fluid transfer device according to claim 10, wherein said coupler comprises:
a first hub having said first cannula extending from one end and a male taper at the opposing end, and
a second hub having said second cannula extending from one end and a female taper at the opposing end, said female taper of said second hub configured to mate with said male taper of said first hub for establishing fluid communication between said first cannula and said second cannula.

12. The fluid transfer device according to claim 11, wherein said first hub and said second hub mate together by a friction fit between said male taper and said female taper.

13. The fluid transfer device according to claim 10, wherein said coupler comprises:
a first hub having a holder end and a connector end, said first cannula extending from said holder end;
a second hub having a holder end and a connector end, said second cannula extending from said holder end; and
a connector having a first end and a second end, said first end of said connector in fluid engagement with said connector end of said first hub and said second end of said connector in fluid engagement with said connector end of said second hub, thereby establishing a path for fluid communication between said first cannula and said second cannula.

14. The fluid transfer device according to claim 13, wherein:
said connector end of said first hub includes a male taper,
said first end of said connector includes a first female taper configured to mate with said male taper of said first hub,
said connector end of said second hub includes a male taper, and
said second end of said connector includes a second female taper configured to mate with said male taper of said second hub.

15. The fluid transfer device according to claim 13, wherein:
said connector end of said first hub includes a female taper,
said first end of said connector includes a first male taper configured to mate with said female taper of said first hub,
said connector end of said second hub includes a female taper, and
said second end of said connector includes a second male taper configured to mate with said female taper of said second hub.

16. The fluid transfer device according to claim 13, wherein said connector is a tube-like structure.

17. The fluid transfer device according to claim 1, wherein said first sampling tube holder region and said second sampling tube holder region are sized so as to accommodate respective first and second sampling tubes having a maximum diameter of about 16 millimeters.

18. A coupler for a fluid transfer device comprising:
a hub including an internal passageway extending between a first end and a second end, said first end of said hub including structure for engagement with a first sampling tube holder having an internal opening capable of accommodating a first sampling tube therein, and said second end of said hub including structure for engagement with a second sampling tube holder having an internal opening capable of accommodating a second sampling tube therein;
a cannula having a first puncture tip extending from said first end of said hub and a second puncture tip extending from said second end of said hub; and
a piercable sleeve extending about at least one of said first puncture tip or said second puncture tip;
wherein said coupler is adapted for establishing interconnection between said first sampling tube holder and said second sampling tube holder with said first puncture tip extending within said internal opening of said first sampling tube holder and said second puncture tip extending within said internal opening of said second sampling tube holder to provide a path for fluid flow between a first sampling tube accommodated within said internal opening of said first sampling tube holder and a second sampling tube accommodated within said internal opening of said second sampling tube holder.

19. The coupler according to claim 18, wherein:
said first end of said hub includes external threads configured to engage internal threads of the first sampling tube holder, and
said second end of said hub includes external threads configured to engage internal threads of the second sampling tube holder.

20. The coupler according to claim 18, further comprising:
a first piercable sleeve extending about said first puncture tip of said cannula; and
a second piercable sleeve extending about said second puncture tip of said cannula.

21. The coupler according to claim 18, wherein said first puncture tip and said second puncture tip of said cannula comprise separate first and second cannulas in fluid communication therebetween.

22. The coupler according to claim 21, wherein said first end and said second end of said hub comprise separate first and second hubs, said first hub having said first cannula extending from one end and a male taper at the opposing end, and said second hub having said second cannula extending from one end and a female taper at the opposing end, said female taper of said second hub configured to mate with said male taper of said first hub for establishing fluid communication between said first cannula and said second cannula.

23. The coupler according to claim 22, wherein said first hub and said second hub mate together by a friction fit between said male taper and said female taper.

24. The coupler according to claim 21, wherein said first end and said second end of said hub comprise separate first and second hubs, said first hub having a holder end and a connector end with said first cannula extending from said holder end, said second hub having a holder end and a connector end with said second cannula extending from said holder end,
said coupler further comprising a connector having a first end and a second end, said first end of said connector in fluid engagement with said connector end of said first hub and said second end of said connector in fluid engagement with said connector end of said second hub, thereby establishing a path for fluid communication between said first cannula and said second cannula.

25. The coupler according to claim 21, wherein:
said connector end of said first hub includes a male taper,
said first end of said connector includes a first female taper configured to mate with said male taper of said first hub,
said connector end of said second hub includes a male taper, and
said second end of said connector includes a second female taper configured to mate with said male taper of said second hub.

26. The coupler according to claim 21, wherein:
said connector end of said first hub includes a female taper,
said first end of said connector includes a first male taper configured to mate with said female taper of said first hub,
said connector end of said second hub includes a female taper, and
said second end of said connector includes a second male taper configured to mate with said female taper of said second hub.

27. The coupler according to claim 21, wherein said connector is a tube-like structure.

28. A method of transferring body fluids between a first sampling tube and a second sampling tube comprising:
a) providing a fluid transfer device comprising first and second sampling tube holders for accommodating first and second sampling tubes therein, said first and second sampling tube holders being joined together through a coupler including a cannula having a first puncture tip and a second puncture tip at opposing ends such that said first puncture tip extends into said first sampling tube holder and said second puncture tip extends into said second sampling tube holder, said second puncture tip including a piercable sleeve extending thereabout;
b) inserting a first sampling tube having a puncturable closure within said first sampling tube holder and puncturing said closure of said first sampling tube with said first puncture tip within said first sampling tube holder; and
c) inserting a second sampling tube having a puncturable closure within said second sampling tube holder and puncturing said closure of said second sampling tube with said second puncture tip within said second sampling tube holder;
wherein fluid flow is established between said first sampling tube and said second sampling tube through said cannula, thereby transferring fluid between said first sampling tube and said second sampling tube.

29. The method of claim 28, wherein said first puncture tip includes a piercable sleeve extending thereabout.

30. The method of claim 29, wherein said step a) comprises:
providing said first and second sampling tube holders for accommodating first and second sampling tubes therein, and
joining said first and second sampling tube holders through said coupler such that said first puncture tip of said cannula extends into said first sampling tube holder and said second puncture tip of said cannula extends into said second sampling tube holder.

31. The method of claim 28, wherein said coupler comprises a first hub and a second hub and said cannula comprises a first cannula comprising said first puncture tip and a second cannula comprising said second puncture tip, said first cannula extending from said first hub and said second cannula extending from said second hub, and wherein said step a) comprises:
providing said first sampling tube holder joined with said first hub with said first puncture tip extending within said first sampling tube holder and providing said second sampling tube holder joined with said second hub with said second puncture tip extending within said second sampling tube holder; and
mating said first hub with said second hub to join said first and second sampling tube holders.

32. A method as in claim 31, wherein said first hub includes a male taper and said second hub includes a female taper, and said mating comprises frictionally fitting said male taper within said female taper.

33. A method as in claim 31, wherein said first hub and said second hub are mated through a connector.

34. A method as in claim 33, wherein said first hub and said second hub include male tapers and said connector includes female tapers at opposing ends thereof, wherein said mating comprises frictionally fitting said male tapers of said first and second hubs within said female tapers at opposing ends of said connector.

35. A method as in claim 33, wherein said first hub and said second hub include female tapers and said connector includes male tapers at opposing ends thereof, wherein said mating comprises frictionally fitting said male taper at opposing ends of said connector within said female tapers of said first and second hubs.

36. A method of assembling a fluid transfer device comprising:
a) providing first and second sampling tube holders for accommodating first and second sampling tubes therein; and
b) joining said first and second sampling tube holders through a coupler including a cannula having a first puncture tip with a first piercable sleeve extending thereabout and a second puncture tip with a second piercable sleeve extending thereabout at opposing ends such that said first puncture tip extends into said first sampling tube holder and said second puncture tip extends into said second sampling tube holder.

37. The method of claim 36, wherein said coupler comprises a first hub and a second hub and said cannula comprises a first cannula comprising said first puncture tip and a second cannula comprising said second puncture tip, said first cannula extending from said first hub and said second cannula extending from said second hub, and wherein said step a) comprises providing said first sampling tube holder joined with said first hub with said first puncture tip extending within said first sampling tube holder and providing said second sampling tube holder joined with said second hub with said second puncture tip extending within said second sampling tube holder; and said step b) comprises mating said first hub with said second hub to join said first and second sampling tube holders.

38. The method of claim 37, wherein said first sampling tube holder is chemically bonded with said first hub and said second sampling tube holder is chemically bonded with said second hub.

39. The method of claim 37, wherein said mating step b) comprises chemically bonding said first hub with said second hub.

40. The method of claim 39, wherein said first hub and said second hub are chemically bonded through a connector.

41. The method of claim 36, further comprising:
c) packaging said first and second sampling tube holders joined through said coupler.

42. The method of claim 41, further comprising:
d) sterilizing said first and second sampling tube holders joined through said coupler.

43. A kit of parts comprising:
a first sampling tube holder having a first open end for accommodating a first sampling tube therethrough and a second end having an opening therethrough;
a second sampling tube holder having a first open end for accommodating a second sampling tube therethrough and a second end having an opening therethrough; and
a coupler for joining said first sampling tube holder and said second sampling tube holder, said coupler comprising a hub having an internal passageway extending between a first end and a second end with a cannula extending from said internal passageway at said first end of said hub to form a first puncture tip with a piercable sleeve extending thereabout and at said second end of said hub to form a second puncture tip with a piercable sleeve extending thereabout, said first end of said coupler including structure for engagement with said second end of said first sampling tube holder to provide said first puncture tip extending through said opening at said second end of said first sampling tube holder and said second end of said coupler including structure for engagement with said second end of said second sampling tube holder to provide said second puncture tip extending through said opening at said second end of said second sampling tube holder.

44. A kit of parts comprising:
a first sampling tube holder having a hollow interior, a first open end for accommodating a first sampling tube within said hollow interior, and a second end supporting a first cannula extending therethrough with a first puncture tip extending into said hollow interior; and
a second sampling tube holder having a hollow interior, a first open end for accommodating a second sampling tube within said hollow interior, and a second end supporting a second cannula extending therethrough with a second puncture tip extending into said hollow interior;
said second end of said first sampling tube holder and said second end of said second sampling tube holder each including structure for engagement therebetween for providing a path for fluid flow between said first cannula and said second cannula.

45. The kit of claim 44, wherein said second end of said first sampling tube holder includes a hollow male tapering surface and said second end of said second sampling tube holder includes a hollow female tapering surface for frictional mating therebetween.

46. The kit of claim 44, wherein said second end of said first sampling tube holder and said second end of said second sampling tube holder include hollow female tapering surfaces, and said kit of parts further comprises:
a hollow connector having a first end with a male tapering surface and a second end with a male tapering surface,
wherein said male tapering surface of said first end of said hollow connector is adapted for frictional mating with said female tapering surface of said second end of said first sampling tube holder and said male tapering surface of said second end of said hollow connector is adapted for frictional mating with said female tapering surface of said second end of said second sampling tube holder, thereby providing a path for fluid flow between said first cannula and said second cannula through said hollow connector.
